(19) United States
(12) Patent Application Publication
(10) Pub. No.: US 2018/0249941 A1
(43) Pub. Date: Sep. 6, 2018

(54) Oculometric Neurological Examination (ONE) Appliance

(71) Applicant: neuroFit, Inc., Mountain View, CA (US)

(72) Inventors: Dorion Liston, Boulder Creek, CA (US); Stephanos Papademetriou, Woodside, CA (US); Rami Ersheid, Mountain View, CA (US); Dmitriy Orlov, San Jose, CA (US)

(21) Appl. No.: 15/604,585

(22) Filed: May 24, 2017

Related U.S. Application Data
(60) Provisional application No. 62/340,996, filed on May 24, 2016.

(51) Int. Cl. A61B 5/00; A61B 3/00; A61B 3/14; A61B 3/113; A61B 5/11

(52) U.S. Cl. CPC A61B 5/4064; A61B 3/0041; A61B 3/0091; A61B 3/145; A61B 3/0008; A61B 3/0083; A61B 2576/02; A61B 3/0025; A61B 5/1128; A61B 5/7221; A61B 5/746; A61B 5/0077; A61B 2560/0475; A61B 3/113

(57) ABSTRACT

The invention concerns observation, measurement and analysis of a person's eye movements while viewing a screen, for purposes of evaluating brain health, eye coordination or level of ability of a person for various pursuits such as sports.

This application claims benefit of provisional application Ser. No. 62/340,996, filed May 24, 2016.

### BACKGROUND OF THE INVENTION

The invention concerns observation, measurement and analysis of a person's eye movements while viewing a screen, for purposes of evaluating brain health, eye coordination or level of ability of a person for various pursuits such as sports.

State-of-the-art approaches in oculomotor physiology use metrics derived from oculomotor behavior (i.e., “oculometrics”) as biomarkers to detect and quantify brain injury and disease. These metrics include measurements common in the physiological literature (e.g., movement latency, acceleration, gain, saccadic duration, saccadic peak velocity) as well as more specialized metrics related to the quality of perceptual processes (e.g., speed-tuning, direction-tuning), or specific oculomotor signs known to occur in disease or injury (e.g., gaze disconjugacy, ocular tremor, pupillary light reflex, squarewave jerk) that are currently used for screening or diagnosis of neurological conditions. The oculomotor literature has characterized qualitative signs that accompany various neurological conditions arising from: degeneration of neural tissue (e.g., Parkinson's disease, Alzheimer's disease, progressive supranuclear palsy, Friedreich's ataxia), idiopathic disease states (e.g., idiopathic elevated intracranial pressure), as well as various types of insults and injury to neural tissue (e.g., traumatic brain injury, cerebral lesions, cerebellar lesions, stroke, alcohol intoxication, hypoxia). Furthermore, these types of oculomotor signs have been reported to occur in a wide variety of psychiatric (e.g., schizophrenia, clinical depression), developmental (e.g., autism), and ophthalmic (e.g., glaucoma, retinitis pigmentosa) conditions. Recent position papers by well-known physiologists and neurologists have advocated for the use of eye-movement-based biomarkers to screen for, detect, and quantify the severity of brain injury and disease (Anderson & MacAskill, 2013; Antoniades, Bak, Carpenter, Hodges, & Barker, 2007; Gorges, Pinkhardt, & Kassubek, 2014; Leigh & Kennard, 2004; Pearson, Armitage, Horner, & Carpenter, 2007; Zee, 2012). As yet, however, no general-purpose neurological device has filled the long-standing unmet need for oculometric examination, despite having been a topic of active investigation for more than a century (Diefendorf & Dodge, 1908).

Qualitative oculomotor exams are used in clinical settings to localize lesions (Bradley, 2004), diagnose vestibular disorders (Leigh & Zee, 2006), and detect cranial nerve palsies (Bradley, 2004), and in field settings to detect alcohol intoxication (Aschan, 1958; Citek, Ball, & Rutledge, 2003), military acute concussion examinations (Coldren, Kelly, Parish, Dretsch, & Russell, 2010), and as part of the gross neurological examination (Bradley, 2004). Although qualitative functional tests of oculomotor behavior are familiar indicators of brain insult, injury or disease in operational and field settings (e.g., police use of gaze paretic nystagmus as an indicator of alcohol intoxication in the field sobriety test) (Stuster & Burns, 1998), state-of-the-art research approaches exploring the quantitative use of eye-movements as biomarkers have not yet overcome a number of technical and personnel barriers to translate these results into a clinically-relevant neurological examination, including: a lack of standardized hardware, lack of standardized oculomotor paradigms, lack of standardized analysis algorithms, proprietary algorithms in commercial eye-tracking software, a high degree of technical skill required to set up and collect high-quality data, complications arising from the complexity of the equipment, expense associated with training personnel in eye-tracking, and limited availability of personnel already trained to collect eye-movement data. To address these issues, we describe a hardware appliance housed in a single enclosure to reduce mechanical degrees of freedom for eye-tracking and ocular measurements, reduce the complexity of setup, and deliver a more portable and robust platform for neurological testing than the current state-of-the art. Furthermore, we describe software modules to automate operation of the hardware device, including: positioning the subject with respect to the hardware appliance, monitoring quality-control during eye-tracking, and automated saving of raw eye images for position traces outside of known ranges. Together, these novel improvements to the state-of-the art achieve an end-product that overcomes barriers in translating oculomotor research results into a useful neurological examination appliance. This appliance can be used as a platform to run standardized oculomotor tests, standardized analysis of eye-position traces, and standardized metrics as summary measurements of oculomotor behavior.

### BACKGROUND ART

The market for medical devices to assess neurological conditions, and associated research and development projects, include a number of purpose-built appliances, devices, or apparatuses used to measure specific neurological signs from ocular measurements. FIG. 1 illustrates the generic set of methodological steps employed by this broad class of devices.

Pupillary responses are a well-known sign of neurological insult or injury (Bradley, 2004; Leigh & Zee, 2006). In the absence of changes in illumination, the pupil also shows relatively high-level effects of visual “target” stimuli of some behavioral significance to the subject (Goldwater, 1972; Hess & Polt, 1960; Privitera, Renninger, Carney, Klein, & Aguilar, 2010) mediated by the sympathetic nervous system (i.e., the fight-or-flight response). The research discipline of pupillometry uses independent variables including: pupil size, dynamics of changes in pupil size, and the pupillary light reflex as measures of dependent variables related to the subject's overall health (e.g., the functional state of the circuitry underlying the pupillary light reflex, the presence or absence of drug intoxication) or the psychological relevance of stimuli to the subject (e.g., as a measure of arousal, relevance, psychological valence). Various patents related to pupillometry methods and devices have been patented (U.S. Pat. No. 5,422,690, U.S. Pat. No. 6,090,051 A, U.S. Pat. No. 7,670,002, US2002/0099305 A1) and products have been designed for the commercial medical device market to measure pupil size, pupillary light reflex, pupil dynamics, and inter-pupillary distance (e.g., Neuroptics, Hoya, Oasis Medical, US Ophthalmic).

Saccadic eye movements are quick flicks of the eye (the English translation of the French word saccade is “jerk” or “flick”) to position the image of a target of interest on a high-acuity region of the retina. In normal subjects, the image of the target of interest is placed on the central portion of the retina, the fovea, with the highest density of cone photoreceptors. Saccadic eye movements follow a stereotyped relationship between peak velocity, amplitude, and duration known as the saccadic main sequence (Bahill, Clark, & Stark, 1975; Leigh & Zee, 2006), which can be used to detect fatigue (Hirvonen et al., 2010; Schmidt, Abel, Dell'Osso, & Daroff, 1979), disease states (e.g., Niemann-Pick type C disease), and alcohol impairment (Leigh & Zee, 2006). Several patents (U.S. Pat. No. 5,422,690, US 2007/0017534 A1, WO2014159498 A2) have been issued on this method and have been incorporated into commercial devices designed for the occupational health market (Pulse Medical Instruments).

Squarewave jerks are small saccadic intrusions during fixation, characterized by a horizontal saccade up to 0.4 degrees, an intersaccadic interval of 200-400 ms, then a second saccade to bring the eye back to its original fixation location (Leigh & Zee, 2006). Several types of neurodegenerative disorders are associated with the occurrence of squarewave jerk, including: progressive supranuclear palsy (Abel, Traccis, Dell'Osso, Daroff, & Troost, 1983; Antoniades, Bak, et al., 2007; Chen et al., 2010), Alzheimer's disease (Kapoula et al., 2014), cerebellar syndromes (Rabiah, Bateman, Demer, & Perlman, 1997), cerebral lesions (Sharpe, Herishanu, & White, 1982), and Friedreich's ataxia (Fahey et al., 2008; Ribai et al., 2007; Spieker et al., 1995). Several methods have been patented for detection of neurological disease, or differential diagnosis of neurological disease using square wave jerk (US2010/0277693 A1, US2012/0238901 A1, US 2016/0022137 A1).

Smooth pursuit eye movements stabilize the moving image of a target of interest on the fovea, and are known to be disturbed in several human disease states, including: schizophrenia (Levin et al., 1988), barbiturate intoxication (Rashbass, 1961), and traumatic brain injury (Heitger, Jones, & Anderson, 2008; Liston, Wong, & Stone, 2016; Suh et al., 2006). The use of abnormalities of smooth pursuit eye movements have been patented to detect physiological impairment from alcohol, drugs, or fatigue (U.S. Pat. No. 8,226,574), to assess drug efficacy (U.S. Pat. No. 9,265,458), to identify cognitive impairment (U.S. Pat. No. 7,819,818), or to identify brain disease or injury. Neural processes supporting motion perception are known to be perturbed in several neurological, psychiatric, and ophthalmic conditions including: retinitis pigmentosa (Turano & Wang, 1992), Alzheimer's disease (Duffy, 2009; Kavcic, Vaughn, & Duffy, 2011; Pelak & Hoyt, 2005), and traumatic brain injury (Heitger et al., 2004; Liston, Wong, et al., 2016; Pelak & Hoyt, 2005), and schizophrenia (Allen, Matsunaga, Hacisalihzade, & Stark, 1990; Hutton & Kennard, 1998; Levin et al., 1988; Levy, Sereno, Gooding, & O'Driscoll, 2010).

The latency of eye movements is defined as the elapsed time between the onset or movement of a visual stimulus and the resulting movement of the eye. Several research reports have documented prolonged latency of saccadic (Kraus et al., 2007; Pearson et al., 2007; Williams et al., 1997) and smooth pursuit (Liston, Wong, et al., 2016) eye movements associated with traumatic brain injury, and prolonged saccadic latency associated with Huntington disease (Antoniades, Altham, Mason, Barker, & Carpenter, 2007), and a purpose-built research device has been patented (U.S. Pat. No. 6,113,237 A) and constructed (Ober Consulting, Poland) to measure saccadic latency (Ober et al., 2003; Pearson et al., 2007), and normative performance metrics have been reported for both pursuit (Schalen, 1980) and saccadic (Bahill, Brockenbrough, & Troost, 1981) eye movements, for a few benchmark tasks.

Gaze disconjugacy is defined as mis-coordination in alignment between the two eyes, has long been known to be a sign of moderate-to-severe brain trauma (Leigh & Zee, 2006) and of neurological disease (Leigh & Zee, 2006). Recent research reports have used gaze disconjugacy as a metric to detect traumatic brain injury (Samadani, Farooq, et al., 2015; Samadani, Ritlop, et al., 2015); several patents (WO 2015023695 A1, WO 2013148557 A1) have been issued on this topic and commercial products have been developed or are being developed by various companies (NeuroKinetics, Oculogica, SyncThink) to measure gaze disconjugacy using a binocular eyetracker.

Following traumatic brain injury, 20-40% of patients report light sensitivity, or photophobia (Zihl & Kerkhoff, 1990), which also occurs in comorbid TBI-PTSD (Goodrich et al., 2014). Measurable signs of photophobia include squinting and blinking, which are grossly observable and straightforward to measure. Several technologies to quantify blinking or squinting have been described, including electromyography (Stringham, Fuld, & Wenzel, 2003) and video-based methods. Furthermore, blink-detection algorithms have been described in medical-device patents (U.S. Pat. No. 5,422,690) are included in proprietary software packages for analysis of eye-position data from commercial eye-trackers (Smart Eye), and have been described in the engineering literature (Espinosa, Roig, Perez, & Mas, 2015).

Routine screening for vestibular disorders includes a set of standardized (S.345-2009) nystagmography tests (ANSI, 2009), including the description of a nystagmograph device. The nystagmograph may use either video-based eye-tracking or skin-mounted electrodes. In various tests, the device tests for: 1) the presence or absence of spontaneous and gaze-evoked nystagmus, 2) the subject's ability to produce fast, accurate, goal-directed saccadic eye movements, 3) whether the subject can track a moving target moving along a predictable path, 4) the presence or absence of positional nystagmus as the subject is either seated in an upright position, or moved from being seated upright to supine, and 5) the presence or absence of nystagmus as one or both lateral semicircular canals are either stimulated with warm water or inhibited with cold water.

Prior art in the neurological literature includes algorithms, methods, and approaches to screen for, identify, or diagnose certain neurological conditions, or impairments, based upon symptoms and signs. For example, based upon the presence or absence of particular oculomotor signs (e.g., ocular tremor, squarewave jerk, prolonged saccadic latency, saccades with low peak velocity), a nested series of if-then statements (US2016/0022137 A1) has been used to identify the presence or absence of certain neurological conditions, by comparing the set of parameters as measured from saccadic eye movements with normalized data (US2007/0017534 A1), or by comparing a series of saccades and fixations to the output of a computational model (US2010/0208205 A1).

In summary, medical devices and medical research instruments built to deliver metrics based upon ocular responses in neurological conditions, include: pupillometers, fitness-for-duty testing stations, electromyography, and monocular or binocular eye-tracking devices, and electrical or optical measurements of squinting or blinking, and, last, algorithms, methods and approaches to assess whether subjects display oculomotor signs of neurological disease.

In addition to the medical devices that measure ocular responses in neurological conditions, the oculomotor research community uses a number of tools that form a body of prior art for this oculometric neurological appliance. First, for decades, research studies have measured oculomotor responses using various techniques, including: photographic methods, circular coils of wire embedded in the sclera or within a contact lens, skin-mounted electrodes, mechanical devices attached to the eye, and video-based optical eye-tracking methods. These devices generate a continuous eye-position signal, for either one eye (monocular eye tracking) or for both eyes together (binocular eye tracking). Second, standardized oculomotor paradigms have been described to allow for a consistent set of stimuli to be presented to patients. Third, standardized analysis methods have been developed to allow for consistent results to be generated from eye-position traces. Fourth, given a standardized task and standardized analysis methods, the development of standardized metrics allows for continuous eye-position traces from one or more trials to be distilled into a set of scalar values that quantify properties of the motor system (e.g., latency, acceleration, gain), the percept of different properties of the stimulus (e.g., speed-tuning, direction-tuning), or sensory-motor transformations (tracking error, catch-up saccade amplitude, threshold value to trigger catch-up saccades).

Research studies investigating oculomotor signs of neurological, psychiatric, and ophthalmic disease in clinical populations typically use standalone, commercial eye-tracking technology. Eye-tracking technology uses some type of photographic, electrical, electromagnetic, or optical measurement appliance to generate continuous eye-position signals (FIG. 1, Prior Art), using various approaches that have been improved and refined over the last century. The first device in this series, Raymond Dodge's “photochronograph”, used a falling photographic plate was used to record a time series record of horizontal eye movements (Diefendorf & Dodge, 1908; Dodge, 1903; Dodge & Cline, 1901). An electrical approach used skin-mounted electrodes to measure muscular activity related to eye movements (Marg, 1951).

An electromagnetic approach used induced currents in circular coils within a magnetic field to record eye position (Robinson, 1963). A mechanical approach used a suction cup adhered to the cornea attached to a stylus to measure two-dimensional eye-position traces (Yarbus, 1967). Next, an electro-optical device used Purkinje images to generate continuous electrical signals (Cornsweet & Crane, 1973). Later, various video-based methods extract locations of eye features from the raw images using, alone or in combination: the pupil centroid, reflection of one or more illuminators off the cornea (1^{st} Purkinje image), the reflection of an illuminator off of the back of the lens (4^{th} Purkinje image), the pupil-iris boundary, iris-sclera boundary, and facial landmarks have been used for eye-tracking, since at least 1980. Numerous patents have been awarded for tracking algorithms based upon combinations of tracked eye features in video images (U.S. Pat. No. 5,231,674 A, U.S. Pat. No. 8,457,352). At present, commercial eye-tracking technology for human studies uses predominantly video-based eye-tracking because of the relatively non-invasive nature of the measurement as compared to other approaches.

Video-based eye-trackers occur in various configurations, many of which have unnecessary mechanical, optical, and software degrees of freedom. The configurations of these tracking devices are optimized for two broad use cases: the “head-fixed” case in which the subject's head is stabilized by either a bite bar or a chin rest, and the “head-free” case in which the subject's head is free to move. Typically, video-based eye-tracking technology consists of one or more digital cameras consisting of a sensor and a lens, an IR light source, and a general-purpose computer or a hardware processing board to analyze images and generate eye-position measurements. For head-fixed tracking, options for standalone trackers include table-mounted hardware for head-fixed eye-tracking (e.g., Arrington, ISCAN, SMI). For head-free tracking, options for standalone trackers include one or more cameras embedded in a housing or frame (Tobii Pro X2-30, LC Technologies The EyeFollower®), one or more cameras mounted independently with desktop hardware (Smart Eye, Face Lab), or an eyetracker embedded in goggles or eyeglass frames for “remote” eye-tracking (Tobii Pro Glasses 2). Commercial eye-tracking devices have been available by Applied Science Laboratories since at least 1982 (ASL, 1982), and ISCAN since at least 1988 (ISCAN, 1988), and several early video-based eye tracking methods were surveyed in 1975 (Young & Sheena, 1975).

Recent position papers in oculomotor physiology have advocated for the use of standardized oculomotor paradigms (Antoniades et al., 2013) to allow for straightforward comparisons. Methodological papers and approaches have offered standardized oculomotor paradigms for pro-saccades (Ober et al., 2003), antisaccades (Antoniades et al., 2013), and step-ramp tracking (Liston & Stone, 2014). In general, methods sections of research reports provide sufficient detail to replicate a specific oculomotor paradigm, although the concept of standardization in oculomotor research and testing has not been as widely adopted as in other clinical visual measurements (e.g., visual perimetry, visual acuity, contrast sensitivity) that have achieved widespread use and implementation in commercial products.

Prior art in the oculomotor literature includes standardized analysis approaches for oculomotor data. These algorithms detect saccades in continuous eye-position traces, segment fixations from saccades, segment a continuous trace into fixations, detect the onset of smooth pursuit. In many cases, these standardized algorithms are proprietary and distributed by hardware vendors as part of a software analysis package (MAPPS by eyesDx, Gaze Tracker by Eyetellect, SmartEye's tracking software by SmartEye, Tobii Pro Studio and Tobii Pro Analytics SDK by Tobii, NYAN 2.0 Analysis Suite by Interactive Minds), although a multitude of such algorithms have been published in the oculomotor literature (Adler, Bala, & Krauzlis, 2002; Daye & Optican, 2014; Konig & Buffalo, 2014; Krauzlis & Miles, 1996; Liston, Krukowski, & Stone, 2013; Liston & Stone, 2014; Tole & Young, 1981).

In summary, prior art in the oculomotor literature includes: eye-tracking technology (in particular, video-based eye-tracking methods in research use since at least 1975 and available in commercial eye-tracking products since at least 1982), standalone video-based eye-tracking technology for head-fixed tracking containing many extraneous degrees of freedom, standardized oculomotor protocols for collection of eye-movement data, and proprietary trade-secret or published algorithms for analysis of eye-movement data.

Despite an extensive catalogue of oculomotor signs of injury and disease known in the oculomotor literature (Leigh & Zee, 2006), the neurological community lacks standardized clinically-relevant tools to assess various aspects of neural processing (Pelak & Hoyt, 2005) especially deployable eye-tracking solutions that can be used in operational, field settings (Liston, Simpson, Wong, Rich, & Stone, 2016; Port, Madsen, Means, T., & Wicks, 2015) such as forward operating hospitals, clinics, and athletic training rooms.

An inherent tradeoff exists between a purpose-built approach optimized for one specific use case and a general-purpose approach which must function for a more general set of use cases. General-purpose approaches necessarily involve more complexity in terms of mechanical, software, and/or hardware degrees of freedom whereas purpose-built approaches can be tailored to the exact specifications of the required task. Limitations of the general-purpose approach include: setup time, complexity, expense, and use of hardware and software with unnecessary degrees of freedom.

Purpose-built approaches, on the other hand, use a more constrained set of hardware or components, optimized for a narrow use case and which function poorly, or cannot function, outside of the constrained use case. Translating the results of clinical oculomotor research into a clinically-relevant oculometric neurological examination has been limited by the availability of trained personnel, resources, and proper eye movement equipment.

Prior art in commercial head-free eye-tracking offers a general-purpose tracking of the head and eye within a given “head box” or “tracking box”. Furthermore, gaze computations based upon 3d measurements (Hennessey & Lawrence, 2013) necessary for head-free tracking yield noisier eye-position measurements than 2d approaches, due to the larger number of measurements of eye features (e.g., multiple corneal reflections, ellipsoidal fit to pupil outline), all subject to independent sources of noise.

Thus, head-free trackers that include one or more cameras embedded in a housing detachable from the visual display (e.g., Tobii Pro X2-30, LC Technologies The EyeFollower®) reduce degrees of freedom relative to trackers with multiple independent cameras (e.g., Smart Eye), and decrease difficulty of mechanical setup somewhat, but retain the disadvantages of head-free tracking (e.g., a larger number of tracked features, computational burden, eye-position noise) for use in collecting high-precision eye-position measurements from clinical populations. Moreover, the complexity of head-free tracking is unnecessary for a clinical device which assumes a known head position and introduces noise needlessly into eye-position traces.

For head-free tracking, the fields of use of general-purpose eye-trackers have been too broad to allow for purpose-built trackers, except in very specialized cases. For example, general-purpose Tobii® eye-trackers are advertised for fields of use including: user experience and interaction, marketing and consumer research, infant and child research, psychology and neuroscience, human performance, education, and clinical research. These devices are intended to generate continuous eye-position traces under the widest possible variety of setup configurations, head positions, and lighting conditions, thus degrading the precision of eye-position measurements as compared with head-fixed tracking.

Prior art in commercial head-fixed eye-tracking solutions (e.g., Arrington, ISCAN, SMI) are designed for general-purpose tracking (e.g., no predefined camera position, no specified display size, no predefined illuminator position, no fixed distance between the subject and the camera), and thus introduce unnecessary complexity for constrained head-fixed tracking (e.g., specified display size, known camera position, known viewing distance). In these systems, cameras and illuminators are all positioned independently from the visual display (for example, see U.S. Pat. No. 8,226,574 FIG. 1), all subject to independent sources of vibration and mechanical noise, and which can all be inadvertently misaligned, degrading the precision of the tracking system and possibly invalidating and/or disturbing the calibration, a drawback also present in eye-tracking systems built for research purposes (Liston, Simpson, et al., 2016). By defining an origin at the center of the display, the location and orientation of each camera can be described in three translational and three rotational degrees of freedom, and by assuming point-source illumination, the location of each illuminator can be described in three translational degrees of freedom, as shown in FIG. 2. These large number of mechanical degrees of freedom increase the complexity of setting up the tracker, make it less robust and less portable, and require data-collection by an operator with some training in eye-tracking. In many cases, setup and optimization of a new standalone eye tracker can take a day or more, making them unworkable in field settings.

For head-fixed tracking, the fields of use have generally been research or clinical applications, for use by highly-trained personnel under controlled, laboratory conditions. Thus, designs using separate components for the illuminator (or illuminators), camera (or cameras), and image-processing hardware have prevailed. The user base for head-fixed tracking technology is assumed to be sufficiently skilled that the set-up, operation, and maintenance of this technology can be regarded as trivial, and the designs also assume that the device will be set up once in a semi-permanent lab or clinical setting, usually on a massive optics table. Thus, the day or more it may require to set up the eyetracker, the complexity associated many mechanical degrees of freedom, and expense associated with general-purpose hardware (e.g., positioners, mounts, zoom lenses, optics hardware) become insignificant compared to the years of use that the hardware may provide in a fixed laboratory setting. Recently, however, the need for field-deployable assessment tools has been noted in the literature (Friedl et al., 2007; Hoyt, Reifman, Coster, & Buller, 2002; Liston, Simpson, et al., 2016).

Whereas the large body of prior art in commercial eye-tracking suffer the limitations of being designed as general purpose products, prior art in neurological assessment technology has been purpose-built to measure a focused set of oculometric signs of neurological disease. For example, prior art in neurological assessment technology using eye-movements has been tied to one sign or a small set of signs, such as: square wave jerk (US2010/0277693 A1, US2012/0238901 A1, US 2016/0022137 A1), pupil diameter (U.S. Pat. No. 5,422,690, U.S. Pat. No. 6,090,051 A, U.S. Pat. No. 7,670,002, US2002/0099305 A1), gaze disconjugacy (WO 2015023695 A1, WO 2013148557 A1, US2015/0051508 A1), saccadic latency (U.S. Pat. No. 5,422,690), saccadic peak velocity (U.S. Pat. No. 5,422,690, US 2007/0017534 A1, WO2014159498 A2), or patterns of fixations and saccades on sets of images (US2010/0208205 A1). Although purpose-built neurological devices that measure a specific oculomotor sign (e.g., pupillometers may measure pupil diameter and dynamics) or a small set of signs (e.g., pupil diameter and saccadic peak velocity), no single purpose-built device may record the entire set of oculometric signs that may occur in a given neurological condition. Thus, a large number of such purpose-built devices may be required to measure the entire set of oculometric signs that may occur in a given neurological condition. Furthermore, prior art in collection of oculometric data requires extensive expertise for setup, programming, and interpretation of data records, all of which may depend on the particular type of equipment being used.

These issues have prevented standardization of oculomotor technology and wide application in medical domains by non-experts.

### BRIEF SUMMARY OF THE INVENTION

The oculomotor research community has recorded an extensive catalog of oculomotor signs of neural disease and injury (Leigh & Zee, 2006). To address need for clinically-relevant assessments of neural function, including motion processing (Pelak & Hoyt, 2005) and for eye-tracking solutions that can be deployed to operational settings (Liston, Simpson, et al., 2016), we describe an oculometric neurological examination (ONE) appliance housed in a single rigid frame, capable of running standardized eye-tracking protocols, standardized oculomotor paradigms, standardized analysis algorithms, and standardized comparisons with normative databases of eye-movement-based metrics. In some embodiments, these protocols, paradigms, algorithms and normative databases may be freely-available online, distributed with publications, or made available by subscription. Some embodiments of this single-frame device provide a general-purpose oculometric solution that is faster to set up, more portable, rugged, and simpler than prior art solutions, in a mechanical sense, while maintaining the measurement quality associated with eye-tracking under laboratory conditions. Embodiments of this device allow for general-purpose measurement of oculomotor signs of disease and injury, providing a standard hardware platform for oculometric research and testing.

### DESCRIPTION OF THE DRAWINGS

FIG. 1 provides a flowchart of methodological steps involved in prior art ocular measurement.

FIG. 2 illustrates a mechanical diagram of components for general-purpose eye-tracking. Solid boxes indicate mechanical components that can be independently positioned. Bi-directional arrows indicate mechanical degrees of freedom, with dashed boxes providing a brief description.

FIG. 3 illustrates a mechanical diagram of components for the first embodiment of our proposed oculometric neurological examination appliance, following conventions of FIG. 2. The visual display, illuminator, and camera consisting of a camera sensor and lens are housed in fixed locations within a single rigid frame. The dashed rectangle illustrates that the hardware device has zero mechanical degrees of freedom.

FIG. 4 shows a perspective drawing of our oculometric neurological examination appliance, showing fixed frame with zero mechanical degrees of freedom. The visual display, centrally-mounted camera, and two offset illuminators are housed in fixed locations within a single rigid frame.

FIG. 5 shows a cutaway drawing of oculometric neurological examination appliance, showing fixed frame with zero mechanical degrees of freedom. The visual display, centrally-mounted camera, and two offset illuminators are housed in fixed locations within a single rigid frame. In this embodiment, the single housing contains a general-purpose PC.

FIG. 6 is a schematic perspective drawing of a subject positioned in front of the oculometric neurological examination appliance, showing the appliance as a single rigid unit.

FIG. 6A is a perspective view similar to FIG. 6, but showing the appliance as two components, with a head engagement device secured to the edge of a desk or table and the visual display or monitor unit positioned on the table.

FIG. 7 illustrates the steps involved in operation of the oculometric neurological assessment appliance. The single unfilled rectangle indicates the general-purpose PC within the single-frame housing. Software modules based upon published methods and procedures are shown as shaded dark gray rectangles. Measurements resulting from each software module are shown as light gray rectangles.

FIG. 8 gives a diagram for quality control computations within one embodiment of the eye-tracking software module. Interchangeable algorithms, models, or comparisons, generically-named, are shown as shaded dark gray rectangles. Sets of resulting measurements are shown as light gray rectangles.

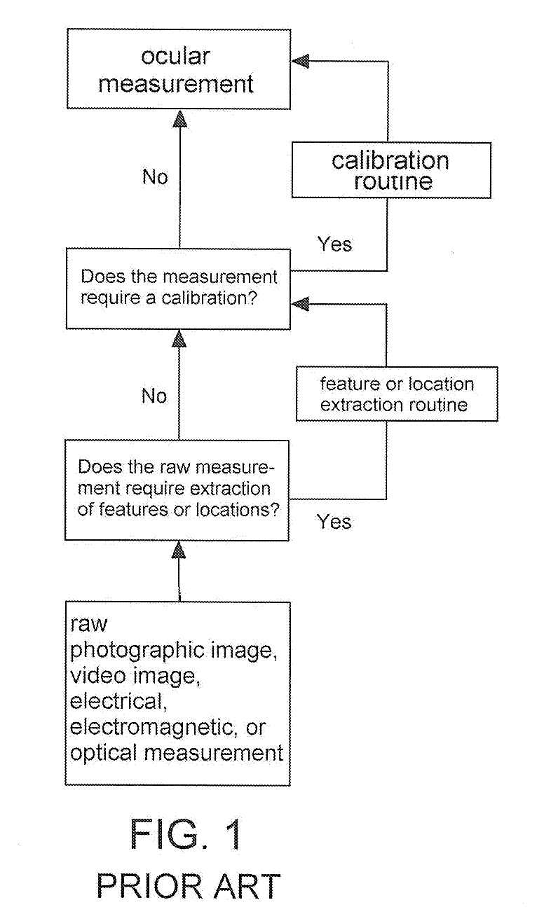

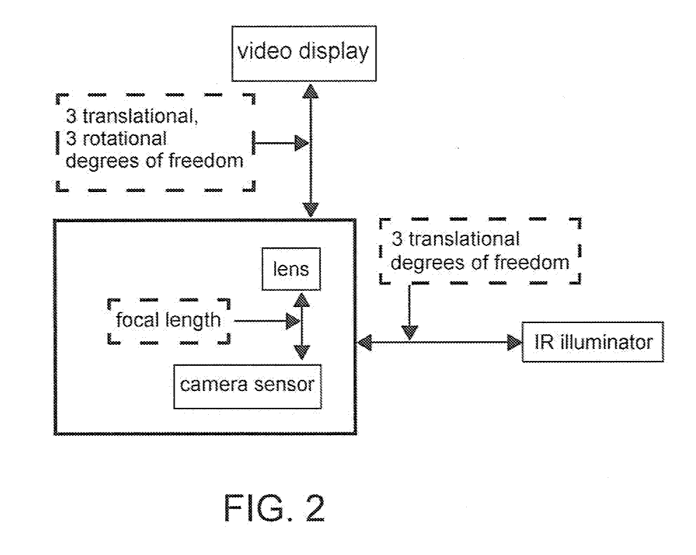

### DESCRIPTION OF PREFERRED EMBODIMENTS

Embodiments of the appliance provide an oculometric solution that is faster to set up, more portable, rugged, and more mechanically-simple than prior art solutions, while maintaining the measurement quality associated with eye-tracking under laboratory conditions. Some aspects of the appliance allow for ease-of-use, software automation, and quality control unavailable in prior art, allowing use by operators without extensive experience or training in eye-tracking or oculomotor physiology. Embodiments of this device allow for general-purpose appliance for measurement and analysis of oculomotor signs of disease and injury, providing a standard hardware platform for oculometric research and testing. The benefits, advantages, and improvements over prior art will become apparent in the attached drawings and description of aspects of the appliance. In practice, this invention allows a user with the skill set of a field medic to make sophisticated medical assessments based upon eye-movement behavior (e.g., measurement of the degree of severity of traumatic brain injury; Wagner, 2017) which heretofore required the skill set of an expert in oculomotor research, behavior, and methods.

### First Embodiment

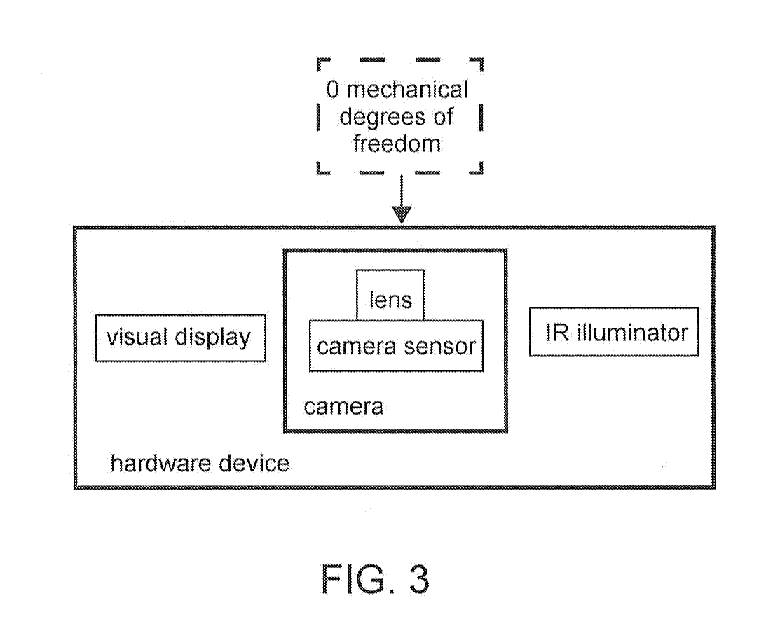

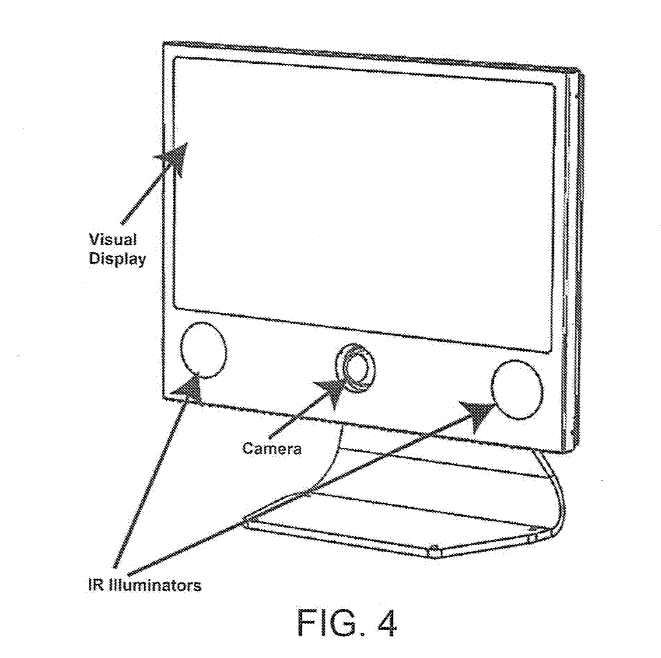

In its first embodiment, the oculometric neurological examination appliance consists of one visual display, one camera consisting of a sensor and lens, and two illuminators (or light sources), and a general-purpose PC, shown diagrammatically in FIG. 3 and in perspective in FIG. 4. A rigid material (e.g., aluminum, steel, high-density plastic, carbon fiber) is used to construct a fixed frame housing a central display, the camera and illuminators. Placement of the camera and light source are optimized for a particular head-fixed tracking situation (e.g., monocular tracking of the left eye, at a viewing distance of 520 mm, with the head aligned horizontally with the center of the display, in controlled primary position).

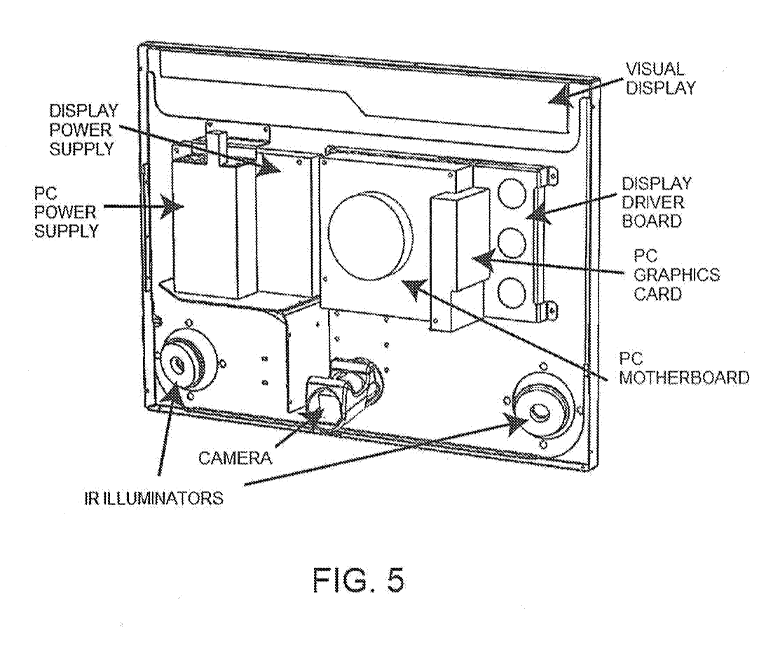

In its first embodiment, the frame has an internal structure, comprised of sub-assemblies that serve to attach, position, or house hardware, optics, or electronic components, including: visual display panel or panels, power supply or power supplies, PC motherboard or boards, PC graphics card or cards, display driver board or boards, camera sensor or sensors, camera lens or lenses, motorized lens assemblies, or other components, shown in a cutaway perspective projection in FIG. 5.

In its first embodiment, the visual display is a 24″ LED-backlit LCD panel with a 16:9 aspect ratio, color, having a resolution of 1080 by 1920 pixels at a refresh rate of 144 Hz; the camera sensor (or, alternatively, image sensor) consists of one ⅔″ monochrome digital sensor, and the lens consists of one 75 mm fixed focal-length lens; the illuminator (or, alternatively, light source) consists of 48 IR-emitting 850-nm LED elements arranged in a circular pattern on a circuit board. The PC consists of a 3.5 GHz CPU, 8 GB of RAM, and a general-purpose motherboard with a 6 Gbps bus speed.

Operation

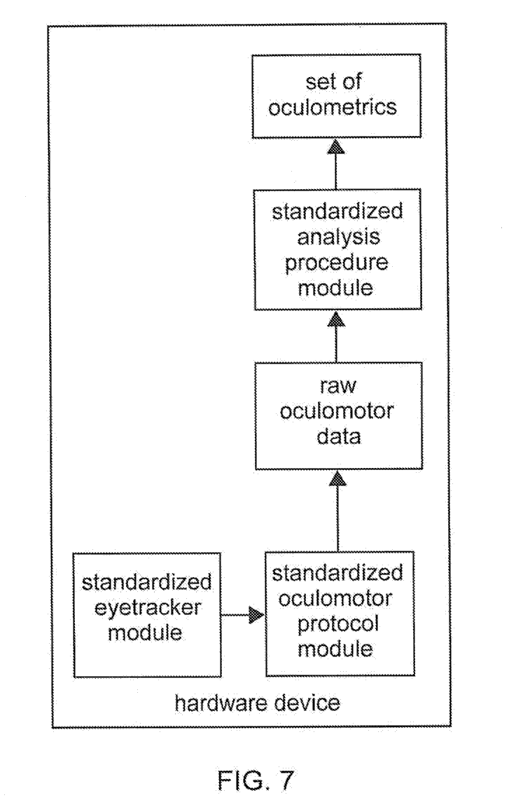

Before operation of our appliance in its first embodiment, software modules for oculometric assessment are first loaded onto computer memory and are run on the CPU of the general-purpose PC, as shown in FIG. 7. Operation involves several steps. First, the subject's head is placed in a fixed location at a viewing distance of 520 mm, as shown in FIG. 6, stabilized with a chin and forehead rest. Second, the eye-tracking module uses a straightforward, well-known two-dimensional dark-pupil tracking algorithm to return the returning the pupil centroid and pupil by applying a threshold brightness value to a monochrome image (Liston, Simpson, et al., 2016), returning the horizontal and vertical location of the pupil center-of-mass, and area. This module remains running for the duration of the subject's interaction with the appliance, and measurement values from the eyetracker module are made available to other software modules on this same machine using a local network connection. Third, the standardized oculomotor protocol module presents visual stimuli to the subject according to standard methods published in the oculomotor literature (e.g., Liston & Stone, 2014). This module saves raw oculomotor data obtained from the eyetracker module to nonvolatile computer memory. Fourth, the raw oculomotor data are analyzed by the standardized analysis procedure module according to standard procedures published in the oculomotor literature (e.g., Liston & Stone, 2014) to generate a set of oculometrics. These oculometrics can be used to detect the presence or absence of neural disease (e.g., Liston, Wong, & Stone, 2016) using likelihood-based metrics (Edwards, 1972) derived from signal-detection theory (Green & Swets, 1966), to quantify the severity those signs while monitoring disease progression or and evaluating treatment outcomes.

### Description and Operation of Alternative Embodiments

In some embodiments, our oculometric appliance includes one or more cameras, one or more light sources, one or more visual displays, one or more general-purpose computers, and/or one or more graphics processing units housed within the single frame.

In some embodiments, the visual display may be of any size, shape, resolution, bit depth, color, aspect ratio, and refresh rate, and may utilize any display technology including: LED-backlit LCD panel, fluorescent tube-backlit LCD panel, purpose-built LED array, LED panel, cathode ray tube, OLED panel, a back-projected laser, a back-projected static or dynamic image, backlit stained glass, or any other static or dynamic display technology.

In some embodiments, the camera sensor may be of any size, including: 1/2.5″, 1/1.8″, ⅔″, 1″, APS-C, APS-H, 35 mm, or medium forma, can be monochrome, grayscale, or color, and can be sampled at any bit depth, including 8-bit or 10-bit. The image sensor elements may have any spectral sensitivity, may be optimized for illumination by infrared light, or may be optimized for illumination by light in any other wavelength range. The image sensor may have a coating to reflect light of particular wavelengths, or may have no such coating.

In some embodiments, the sampling rate of the camera sensor may be different than 250 Hz. In some embodiments, the sampling rate may be greater than 250 Hz, including: 500 Hz, 1000 Hz, or any other sampling rate. In other embodiments, the sampling rate of the camera may be 60 Hz, 120 Hz, 240 Hz, or any sampling rate slower than 250 Hz.

In some embodiments, the camera lens may have motorized components, may be fixed, or may be capable of manual adjustment; the camera lens may be of any focal length, may be a wide-angle, telephoto, perspective-control, or macro lens; the camera lens may be equipped with an IR-pass filter to block visible light, a band-pass filter for some other wavelength range, or it may have no such filter.

In some embodiments, the illuminator may consist of light-emitting elements that emit any wavelength including: 850 nm, 940 nm, monochromatic light in the visible spectrum, or a plurality of wavelengths in the UV, IR, visible spectra; the illuminator may include elements of one uniform wavelength (or wavelength range), or may include two or more types of elements, each emitting one wavelength (or wavelength range); the light-emitting elements in the illuminator may be LED, incandescent, fluorescent, or any other lighting technology; any number of light-emitting elements may be grouped together, in any shape or pattern, to form an illuminator and may be constructed to be driven as an ensemble or individually; the illuminator may be constantly powered to achieve maximum brightness, or may be dimmable.

In some embodiments, one or more input devices may be added to the device (e.g., a joystick, button box, response pad, gamepad, keyboard, or computer mouse).

In some embodiments, the subject may be positioned at a viewing distance other than 520 mm. 520 mm is a standard, commonly-used viewing distance, at which distance one cm equals one degree of visual angle. For some visual applications (e.g., visual perimetry, optokinetic nystagmus), more eccentric measurements are possible with closer viewing distances, to allow for the display to occupy the largest possible extent of the subject's visual field. For other visual applications (e.g., visual acuity measurements), finer-grained measurements are possible with farther viewing distances.

In some embodiments, the head of the subject may be rotated about the nasal-occipital axis to elicit torsional eye movements.

In some embodiments, the rigid geometry provided by device frame allows the appliance to be used to make anthropometric measurements, in one tracking mode for the device. By a “relatively low sampling rate”, is meant 60 Hz or lower using the entire field of view of one camera. The device could be used to measure: interpupillary distance, pupil diameter, shape and curvature of eyelids, reflectance properties of skin, sclera, eyelashes, or other facial features under IR or visible light, distance between facial features, head size, ocular range of motion, radius of curvature of the cornea, radius of curvature of the globe, the opacity of the cornea or lens, ocular misalignment, relative pupillary brightness responses of both eyes, and other facial or ocular measurements. Given a sufficiently-bright, high-resolution visual display (e.g., LED, OLED), and two or more cameras, reflections of individual pixels (or small groups of pixels) off of the cornea could be used to measure corneal shape.

In some embodiments, the combined stimulus-display and eye-tracking capability provided by the appliance frame allows the appliance to be used to make standard visual or neuro-ophthalmic measurements, including: visual acuity, static or dynamic visual field measurements (Schiller et al., 2006), eyelid droop (ptosis), ocular deviation, stability of fixation, range of eye movement, gaze-evoked nystagmus, pupillary defects, optokinetic nystagmus.

In some embodiments, additional software modules may be run on the appliance, or networked machine or a central data server, including a “comparison with normative databases” module, to quantify the similarity between a single set of oculometrics and corresponding sets of metrics for control and disease populations (e.g., Liston, Wong, & Stone, 2016). By “normative database”, is meant a set of “standardized oculometrics” (e.g., the set of 10 metrics as described by Liston & Stone, 2014) from a known, documented reference population (e.g., subjects with Traumatic Brain Injury in the mild-to-moderate range) on a “standardized oculomotor protocol” (e.g., step-ramp tracking as described by Liston & Stone, 2014). In one instance, this statistical comparison is framed as an ideal-observer detection problem (Green & Swets, 1966), in which the task is to assign the measured set of oculometrics to one of two categories, control or disease. In this instance, the signal to be detected is the presence of oculometric signs consistent with “disease”. In other instances, the comparison may be made to two or more disease populations, in addition to a control population. By constructing a “template” for each disease, referenced to the median and variance in the normal population, a likelihood metric can be constructed to quantify the similarity between a given set of oculometrics and each disease “template” (Edwards, 1972; Green & Swets, 1966). In other instances, the “comparison with normative databases” module may use a logical series of “if-then” statements for differential diagnosis, a checklist of signs or symptoms above a given magnitude, the presence or absence of a specific oculomotor sign, some combination of the above methods, or another published or unpublished method of comparison.

In some embodiments, the standardized eyetracker module may use a tracking algorithm that uses features other than, or in addition to, the dark pixels in the pupil, such as the boundary between the pupil and iris, the boundary between the iris and the sclera, the outline of eyelids, or corneal reflections, in one tracking mode run at a relatively high sampling rate. By “relatively high sampling rate” is meant 120 Hz or higher for operational eye-tracking. These features may be estimated by thresholding pixels according to the steepness of the luminance gradient, a luminance threshold, some combination of both, or other feature-extraction techniques.

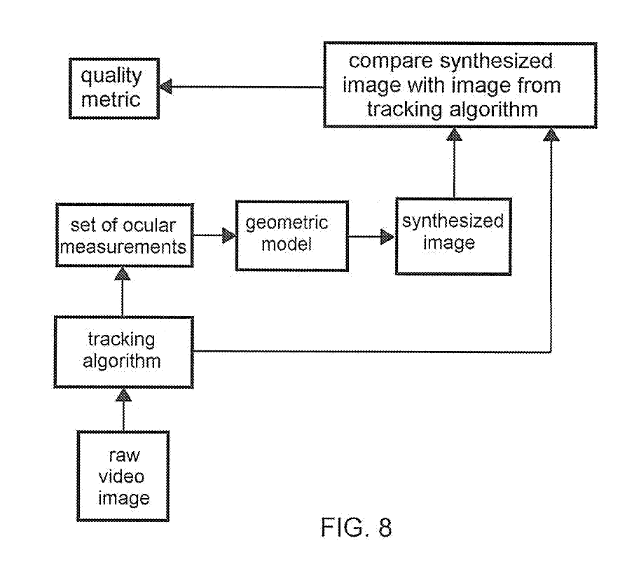

In some embodiments, the standardized eyetracker module may contain a further quality-control computation, shown diagrammatically in FIG. 8, for quality metrics. By “quality metrics”, is meant a normalized cross correlation between an image of the eye and an image synthesized from an ideal geometric model of the eye (e.g., a circular pupil) that meets or exceeds a threshold of 0.85, or 85%. In these embodiments, the tracking algorithm being used will be linked to a geometric model. In one embodiment in which the tracking algorithm returns a set of three ocular measurements (i.e., the horizontal and vertical locations of the pupil center of mass and pupil area) the geometric model will synthesize an image containing a circular pupil for comparison. In another embodiment in which the tracking algorithm returns a set of five ocular measurements (i.e., five parameters describing the elliptical boundary between the pupil and the iris) the geometric model will synthesize an image containing an elliptical pupil for comparison. In other embodiments, synthesized images may contain arbitrary shapes or features defined by the set of ocular measurements, as the geometric model returns an ideal, noise-free image. If the raw video image were completely noise-free and conformed to the model geometry and implicit assumptions of the “geometric model”, then the tracking algorithm would contain an intermediate image that is a pixel-for-pixel match to the synthesized image.

In some embodiments, one or more of the images used in, or returned by, the tracking algorithm may be compared to the synthesized image using cross-correlation. In other embodiments, the comparison may be based upon addition, subtraction, multiplication, cross-correlation, normalized cross-correlation, division, or some other computation.

In some embodiments, the standardized eyetracker module may be constructed to save the raw images recorded from one or both eyes during each trial into a ring buffer, to be saved to disk upon receiving a “save” command from another module, a “self-archiving” feature. By “self-archiving”, is meant the ability to store a sequence of raw, unprocessed video images of the eye or eyes whilst performing an oculomotor behavior. As the raw video images from patient subjects may contain heretofore undocumented patterns of eye movement behavior, the raw image data from this neurological appliance may prove useful for further study and analysis given that abnormal trials can be identified as the subject completes the task. Furthermore, having the set of raw images recorded from a particular trial exhibiting abnormal eye-position measurements may rule out the presence of some algorithmic artifact in the eyetracker software module, and allow the physiological characteristics of certain states of brain disease to be more completely documented.

In some embodiments, the standardized oculomotor protocol module will generate some number of trials of Rashbass (1961) step-ramp motion. By “standardized oculomotor protocol”, is meant a predefined recipe for a sequence of stimuli used to elicit oculomotor behavior such as smooth pursuit or saccadic eye movements, as specified in a publication (e.g., Liston & Stone, 2014 describing step-ramp motion that varies unpredictably in onset timing, speed, and direction). In the first embodiment, the number of step-ramp motion trials is 180; the position step is fixed at 200 ms; the direction is uniformly distributed around the unit circle (0°-358°, in 2° increments); the speed is uniformly sampled from 16-24 deg/s, in steps of 2 deg/s; and onset timing is sampled from an exponential distribution (200 ms to 5000 ms, with a mean of 700 ms). In other embodiments, the standardized oculomotor protocol module may replicate published methods in the oculomotor literature, including: eye-position calibration (Beutter & Stone, 2000; Liston, Simpson, et al., 2016), antisaccades (Antoniades et al., 2013; Crevits, Hanse, Tummers, & Van Maele, 2000; Heitger et al., 2004), visually-guided saccades (Heitger et al., 2004), memory-guided saccades (Crevits et al., 2000; Heitger et al., 2004), gap saccades (Drew et al., 2007; Krauzlis & Miles, 1996), step-ramp tracking (Liston & Stone, 2014; Rashbass, 1961; Robinson, Gordon, & Gordon, 1986), tracking of predictable sinusoidal motion (Heitger et al., 2004), tracking of predictable circular motion, tracking of unpredictable sum-of-sines motion. In other embodiments, the standardized oculomotor protocol module may describe a novel stimulus of neurological or oculomotor interest.

In some embodiments, the standardized analysis procedure software module may consist of a set of published methods, processes, or algorithms to distill raw oculomotor data into a set of oculometrics. By “standardized oculometric analyses”, is meant a documented set of algorithms applied to eye-position traces to result in a set of a set of “standardized oculometrics”. For a step-ramp stimulus for example, the onset of smooth pursuit is measured using a best-fit linear “hinge” model having baseline duration of 100 ms, over the period from 100 ms to 400 ms following the onset of motion; saccades are detected by convolving the radial velocity trace along the direction of the stimulus with a saccade-shaped velocity template, and registering a saccade when the energy exceeds one degree; pursuit direction is measured by measuring the average direction of saccade-free pursuit over the interval from 400 ms to 700 ms following the onset of motion; pursuit direction error is measured by subtracting stimulus direction from pursuit direction; pursuit speed is measured by measuring the average speed of saccade-free pursuit over the interval from 400 to 700 ms following motion onset; pursuit speed error is measured by subtracting stimulus speed from pursuit speed; and speed responsiveness is measured by finding the slope of linear regression of pursuit speed against stimulus speed. In other embodiments, the standardized analysis procedure software may be used experimentally to test the efficacy of unpublished methods, processes, or algorithms under development.

In some embodiments, the set of “standardized oculometrics” returned by the standardized oculometric analyses may include a set of metrics that documents an aspect of functional neurological performance, as assayed by a “standardized oculomotor protocol”. By “standardized oculometrics” is meant a documented set of measurements that can be extracted from a “standardized oculomotor protocol” using “standardized oculometric analyses”, for example the set of measurements including: smooth pursuit latency, smooth pursuit acceleration, steady-state gain, catch-up saccade amplitude, proportion of smooth movement, direction-tuning noise, speed-tuning responsiveness, and speed-tuning noise are “standardized oculometrics” for the step-ramp tracking instance of a “standardized oculomotor protocol”. In other instances, standardized oculometrics may include, for example: the direction of smooth-pursuit tracking, smooth-pursuit direction error, smooth pursuit latency, smooth pursuit acceleration, catch-up saccade amplitude, accumulated eye-position error triggering catch-up saccades, smooth pursuit velocity, smooth pursuit velocity error, smooth pursuit velocity gain, smooth pursuit velocity responsiveness, the proportion of tracking movement consisting of smooth pursuit, saccadic latency, saccadic duration, saccadic peak velocity, the velocity trajectory of saccadic eye movements, saccadic amplitude, saccadic amplitude error, saccade direction, saccade direction error, saccade curvature, the proportion of saccades toward a bright stimulus, the proportion of saccades away from a bright stimulus, the effectiveness of a cue giving the likely location of an upcoming target, or others. In other instantiations, other published or unpublished quantities of clinical or physiological interest may be generated, extracted from eye-position traces, pupillary responses, eye reflectance measurements, or other ocular, facial, or anthropometric measurements. In other embodiments, this appliance may be used experimentally to test the efficacy of unpublished oculometrics.

In some embodiments, our oculometric neurological examination device may use interchangeable software modules based upon the methods, descriptions, analyses, and data in published reports. In one embodiment, these software modules include: an eyetracker module (Liston, Simpson, et al., 2016), a standardized oculomotor protocol consisting of step-ramp motion (Liston & Stone, 2014), an standardized oculometric analysis procedure (Liston & Stone, 2014), and a comparison with normative databases module (Liston, Wong, et al., 2016). In another embodiment, these software modules include: an eyetracker module (Liston, Simpson, et al., 2016), a standardized oculomotor protocol consisting of anti-saccades (Antoniades, Bak, et al., 2007), a saccade-detection analysis procedure (Liston et al., 2013), and compared with normative databases for traumatic brain injury (Heitger et al., 2004; Kelley, Ranes, Estrada, & Grandizio, 2015). This novel, modular design yields a general-purpose neurological device to measure any combination of eye-movement-based metrics (“oculometrics”) that can be generated from standardized oculomotor protocols and quantified with standardized analysis packages, given the hardware and software constraints of the appliance.

In some embodiments, some or all software modules may be run on a networked machine or a central data server, rather than on the appliance.

In some embodiments, the single-housing oculometric design provides a highly-constrained geometry for monocular applications, improving upon conventional eye-tracking approaches by allowing the spatial layout of the camera, the camera sensor, and the lens to be tailored for monocular tracking, then permanently fixed. For example, for the 2012 U.S. Army population, the anthropometric range of interpupillary distances (IPD) spans 5.6 cm (1^{st} percentile) to 7.25 cm (99^{th} percentile), with a median of 6.4 cm, while the corresponding range in females spans 5.35 cm (1^{st} percentile) to 7.05 cm (99^{th} percentile), with a median of 6.2 cm. Thus, for monocular tracking of the left eye, a relatively small, inexpensive sensor horizontally offset leftward by 3.15 cm effectively splits the difference between the range of expected left-eye positions ((7.25/2+5.35/2)/2=3.15 cm). Given constraints of the tracking solution (viewing distance: d=520 mm; pupil translation: g=12 mm; expected side-to-side head translation: m=10 mm), the 3.5-degree horizontal field of view may be computed analytically.

$${FOV}_{horizontal} = arctan ( \frac{( {OS}_{max} - {OS}_{min} ) + g + m}{d} )$$

$${FOV}_{horizontal} = arctan ( \frac{( \frac{72.5}{2} - \frac{53.5}{2} ) + 12 + 10}{520} ) = 3.5 deg$$

In some embodiments, the single-housing oculometric design provides a highly-constrained geometry for binocular tracking applications. Given constraints mentioned above (viewing distance: d=520 mm; pupil translation: g=12 mm; expected head translation: m=10 mm), for the same 2012 Army population, a 10.3-degree horizontal field of view may be computed analytically for a single, central sensor.

$${FOV}_{horizontal} = arctan ( \frac{{IPD}_{max} + g + m}{d} )$$

$${FOV}_{horizontal} = arctan ( \frac{72.5 + 12 + 10}{520} ) = 10.3 deg$$

In some embodiments, a motor may be added to position the subject's eye in an identical vertical location with respect to the center of the monitor, to account for the range of anthropometric variability in the location of the eyes within the subject's head. These and other objects, advantages and features of the invention will be apparent from the following description of a preferred embodiment, considered along with the accompanying drawings.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. An oculometric neurological examination apparatus, comprising:
a display console including a screen to present moving or stationary objects to be viewed by the subject, at least one illuminator directed toward at least one eye of the subject, and a camera directed toward a position a subject's eyes will assume when the subject is engaged with the apparatus,
electronics connected to receive images from the camera and produce signals representing eye movements of the subject, and
the camera of the console being at a fixed position and distance from the position to be assumed by a subject's eyes, such that the camera will be focused on the eyes of a subject to be examined.

2. The apparatus of claim 1, wherein the display console includes the camera and illuminator in a common housing, the housing being physically connected to the head with a band or strap.

3. The apparatus of claim 1, wherein the display console includes the camera and the illuminator in a common housing, and the apparatus including a frame having at one end a head contact on which the subject's head can rest in a fixed position, and at an opposite end the display console.

4. The apparatus of claim 1, wherein the display console has a base adapted to sit on a flat surface at a known position and the console with the camera and illuminator is fixedly positioned relative to said flat surface such that the camera is at said fixed position and distance from the eyes of a subject to be examined.

5. The apparatus of claim 1, wherein the camera has a lens of the size and focal length to allow the apparatus to capture images from one or both eyes of a subject, spanning the entire anthropometric range of inter-pupillary distance for adult humans.

6. The apparatus of claim 1, including a chin or forehead rest holds that holds a subject's head still such that the subject's eyes are at said fixed position and distance relative to the camera.

7. The apparatus of claim 1, wherein said distance is in the range of about 50 to 60 cm.

8. The apparatus of claim 1, wherein the electronics include processing means for receiving the signals representing eye movements, for analyzing data from the signals and for comparing the data to benchmark data indicating normal or specific abnormal conditions.

9. The apparatus of claim 8, wherein the processing means further includes a quality control feature to assure signal quality by comparing camera image frames collected to a preselected geometric model of a human eye to ensure that images collected by the camera conform to a minimum quality standard, the feature providing an alert to a user or to the subject if signals fall below the minimum quality standard, indicating the signal data are inaccurate.

10. The apparatus of claim 8, wherein the processing means has means for operating a plurality of different tracking modes, including an initial mode using the full field of view of the camera, at a relatively low sampling rate, in order to locate features of the subject's head and eyes, and an operational tracking mode wherein the electronic means samples a sub-region of the camera field of view at a much higher sampling rate for measurement of eye-movement behavior.

11. The apparatus of claim 8, wherein the electronic means includes means for comparing recorded oculometric data from camera images with corresponding data recorded from any of a plurality of different reference populations having different characteristics.

12. The apparatus of claim 8, wherein the electronic means has means to perform a plurality of different standardized analyses on oculometric data collected.

13. The apparatus of claim 1, wherein the electronics include comparison means for making self-referenced comparisons, wherein the signals can be compared corresponding measurements recorded previously from the same subject.

14. The apparatus of claim 1, further including a self-archiving means for saving a series of unprocessed images from the camera to a storage medium in the event that an unusual form of eye movement is detected in the subject.

15. An oculometric neurological examination apparatus, comprising:
a display console including a screen to present moving or stationary objects to be viewed by the subject,
at least one illuminator directed toward at least one eye of the subject,
a camera directed toward a position a subject's eyes will assume when the subject is engaged with the apparatus, the camera being at a fixed position and distance from the position to be assumed by a subject's eyes such that the camera will be focused on the eyes of a subject to be examined,
first electronic means for receiving images from the camera and produce signals representing eye position or movement,
second electronic means for computing a plurality of quality metrics from the analyzed eye images or eye position or movement signals,
third electronic means for performing a plurality of standardized oculomotor protocols,
fourth electronic means for performing a plurality of standardized oculometric analyses on the data collected,
fifth electronic means for generating a plurality of standardized oculometrics from the data and analyses, and
sixth electronic means for comparing oculometrics to oculometrics from normative databases.

16. The apparatus of claim 15, including a chin or forehead rest that holds a subject's head still such that the subject's eyes are at said fixed position and distance relative to the camera.

17. The apparatus of claim 15, wherein the display console includes the camera and the illuminator in a common housing, and are physically connected to the head with a band or strap.

18. An oculometric neurological examination apparatus, comprising:
a frame having at one end a chin rest and a forehead rest for a subject to be examined,
at an opposite end of the frame, a display console including a screen to present moving objects to be viewed by the subject, at least one illuminator directed toward at least one eye of the subject, a camera directed toward a position a subject's eyes will assume when the object is engaged on the chin est and forehead rest, and electronics to receive images from the camera and produce signals representing eye movements of the subject,
the camera of the console being at a fixed position and distance from the position to be assumed by a subject's eyes, such that the camera will be focused on the eyes of a subject to be examined.

19. The apparatus of claim 18, wherein the said distance is in the range of about 50 to 60 cm.

20. The apparatus of claim 18, wherein the electronics include means for receiving the signals representing eye movements, for analyzing data from the signals and for comparing the data to benchmark data indicating normal or specific abnormal conditions.
